(19) United States
(12) Patent Application Publication
(10) Pub. No.: US 2008/0031929 A1
(43) Pub. Date: Feb. 7, 2008

(54) LIQUID BANDAGE

(76) Inventor: Richard W. Baggett, Bountiful, UT (US)

Correspondence Address: KIRTON & McCONKIE;A PROFESSIONAL CORPORATION, 1800 EAGLE GATE TOWER, 60 EAST SOUTH TEMPLE STREET, SALT LAKE CITY, UT, 84111, US

(21) Appl. No.: 11/768,709

(22) Filed: Jun. 26, 2007

Related U.S. Application Data
(63) Continuation-in-part of application No. 11/191,841, filed on Jul. 28, 2005.

(51) Int. Cl. A61K 31/765; A61K 9/70; A61P 17/02

(52) U.S. Cl. 424/443; 424/78.37

(57) ABSTRACT

The present invention relates to hemostatic fabric materials, and to the methods for making and using such materials. In particular, the present invention relates to hemostatic fabric materials made from chemically treated plant materials that are soluble on wound surfaces.

### RELATED APPLICATIONS

This application is a continuation in part of U.S. patent application Ser. No. 11/191,841, filed Jul. 28, 2005, entitled “Hemostatic Material”.

### BACKGROUND OF THE INVENTION

1. Field of the Invention

The present invention relates to hemostatic gel that is bioabsorbable, which can be fabricated into a variety of forms suitable for use in controlling bleeding from a variety of wounds and to methods for making and using the same.

2. Background and Related Art

Surgical procedures and injuries are often characterized by blood loss. Conventional approaches for dealing with blood loss, such as manual pressure, cauterization, or sutures can be time consuming and are not always effective in controlling bleeding.

A number of topical hemostatic agents have been developed to control bleeding resulting from surgical procedures and injury. Some hemostatic agents, such as collagen-based powders, sponges, and cloths, are of a particulate nature. Particulate hemostatic agents provide a lattice for natural thrombus formation, but are unable to enhance this process in coagulopathic patients. Pharmacologically-active agents such as thrombin can be used in combination with a particulate carrier, for example, as in a gelfoam sponge or powder soaked in thrombin. Thrombin has been used to control bleeding on diffusely bleeding tissue surfaces, but the lack of a framework onto which the clot can adhere has limited its use. The autologous and allogenic fibrin glues can cause clot formation, but do not adhere well to wet tissue and have little impact on actively bleeding wounds.

Accordingly, a hemostatic fabric material, which enhances the process of coagulation is desirable. However, currently known hemostatic fabric materials as used around the world are insoluble and have the following deficiencies: they can not be used inside the body because absorption is slow and incomplete; additional medicine is usually needed to achieve the hemostasis efficacy; pain usually results when the material is removed; and they effect slow hemostasis. Therefore, improved hemostasis materials are still needed in modern medical treatments.

Accordingly, a hemostatic material that is bioabsorbable, which provides superior hemostasis, and that can be fabricated into a variety of forms suitable for use in controlling bleeding from a variety of wounds is desirable.

### SUMMARY OF THE INVENTION

The present invention relates to a hemostatic gel that is bioabsorbable, which can be fabricated into a variety of forms suitable for use in controlling bleeding from a variety of wounds and to methods for making and using the same. In particular, the present invention relates to hemostatic fabric materials made from chemically treated plant materials that are soluble on wound surfaces. The hemostatic materials can be combined with a liquid to form a hemostatic gel. The hemostatic material and gel are suitable for controlling active bleeding and oozing.

The current invention provides a hemostatic gel, which after chemical treatment by the method of the invention is soluble both outside and inside the body so that the material can be absorbed by the human body. In addition, the gel has the following advantages: no other medicine is needed in the material or gel, hemostasis is fast, the material is easy to carry and store, the gel can meet the requirements of surgery and daily use, the gel can be applied for emergent hemostasis in the battle ground, the gel causes no pain and can match wounds accurately, there are no side effects, and the gel exhibits high hemostasis efficacy even to patients with blood-coagulation obstruction. The hemostatic gel of the invention is simple, easy to use, economical, can be utilized under any circumstances where hemostasis is needed, and can be made economically in the industry.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates the chemical formula of a compound of which the hemostatic material of the invention is comprised.

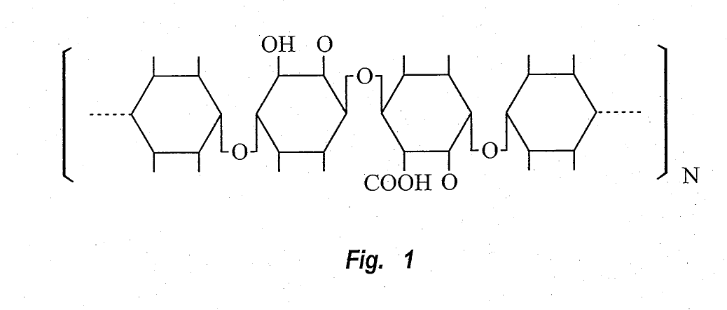

### DETAILED DESCRIPTION OF THE INVENTION

The present invention relates to a hemostatic material that is bioabsorbable, which can be fabricated into a variety of forms suitable for use in controlling bleeding from a variety of wounds and to methods for making and using the same. In particular, the present invention relates to hemostatic fabric materials made from chemically treated plant materials that are soluble on wound surfaces. The hemostatic materials are suitable for controlling active bleeding and oozing from tissues.

1. Hemostasis

Hemostasis is the mechanism (e.g., normal vasoconstriction, abnormal obstruction, coagulation, or surgical means) that stems bleeding after injury to the vasculature. Biological hemostasis depends on both cellular components and soluble plasma proteins. In particular, hemostasis by coagulation is dependent upon a complex interaction of plasma coagulation and fibrinolytic proteins, platelets, and the blood vasculature. The hemostatic process may be conceptually separated into three stages: primary hemostasis, secondary hemostasis, and tertiary hemostasis.

Primary hemostatsis is principally characterized by the formation of a primary platelet plug. The plug is formed as circulating platelets adhere and aggregate at sites of blood vessel injury. In areas of high shear rate (e.g., microvasculature) aggregation is mediated by von Willebrand factor (vWf), which binds to glycoprotein Ib-IX in the platelet membrane. In areas of low shear rate (e.g., arteries) fibrinogen mediates the binding of platelets to the subendothelium by attaching to a platelet receptor. Aggregation beings with platelets adhering to exposed subendothelium. When platetets adhere to the vessel wall they change shape and activate the collagen receptor on their surface to release alpha and dense granule constituents. Injury to the blood vessel wall is additionally followed by vasoconstriction. Vasoconstriction not only retards extravascular blood loss, but also slows local blood flow, enhancing the adherence of platelets to exposed subendothelial surfaces and the activation of the coagulation process.

Formation of the plug is followed by an aggregation response. Activation of platelets results in exposure of anionic phospholipids that serve as platforms for the assembly of blood coagulation enzyme complexes. Platelet aggregation involves the activation, recruitment, and binding of additional platelets to the adhered platelets. Aggregation is promoted by platelet agonists such as thromboxane 2, PAF, ADP, and serotonin. Activated platelets synthesize and release thromboxane and platelet activating factor, which are potent platelet aggregating agonists and vasoconstrictors. Activation is enhanced by the generation of another platelet agonist, thrombin, through the coagulation cascade. Platelet aggregation is mediated primarily by fibrinogen, which binds to glycoprotein IIb/IIIa on adjacent platelets. This aggregation leads to the formation of the primary platelet plug, and is stabilized by the formation of fibrin.

Secondary hemostasis is characterized by fibrin formation through the coagulation cascade, which involves circulating coagulation factors, calcium, and platelets. The coagulation cascade involves three pathways: intrinsic; extrinsic; and common. The main pathway for initiation of coagulation is the extrinsic pathway, while the intrinsic pathway acts to amplify the coagulation cascade.

The extrinsic pathway involves the tissue factor and factor VII complex, which activates factor X. The extrinsic pathway of blood coagulation is initiated when blood is exposed to tissue factor. Tissue factor, a transmembrane protein, is expressed by endothelial cells, subendothelial tissue and monocytes, with expression being upregulated by cytokines. Tissue factor binds activated factor VII (factor VIIa) and the resulting complex activates factors X and IX. Factor X, in the presence of factor V, calcium, and platelet phospholipid, then activates prothrombin to thrombin. This pathway is rapidly inhibited by a lipoprotein-associated molecule referred to as tissue factor pathway inhibitor. However, the small amount of thrombin generated by this pathway activates factor XI of the intrinsic pathway, which amplifies the coagulation cascade.

Thrombin activates the intrinsic pathway by activation of factors XI and VIII. In the intrinsic pathway activated factor IX (factor IXa) combines with factor VIIIa to provide a second means to activate factor X. The intrinsic pathway involves high-molecular weight kininogen, prekallikrein, and factors XII, XI, IX and VIII. Factor VIII acts as a cofactor (with calcium and platelet phospholipid) for the factor IX-mediated activation of factor X. Activated factor IX, together with activated factor VIII, calcium, and phospholipid, referred to as tenase complex, amplify the activation of factor X, generating large amounts of thrombin.

The extrinsic and intrinsic pathways converge at the activation of factor X. The common pathway involves the factor X-mediated generation of thrombin from prothrombin (facilitated by factor V, calcium and platelet phospholipid), with the production of fibrin from fibrinogen. Factor Xa complexes with factor Va and prothrombin to form prothrombinase, which cleaves prothrombin to generate thrombin, the key enzyme in hemostasis. In the final step of the coagulation cascade, thrombin cleaves fibrinogen to generate fibrin monomers, which then polymerize. This polymer is covalently cross-linked by factor XIIIa (itself generated from factor XIII by thrombin) to form a chemically stable clot. Thrombin also feeds back to activate cofactors V and VIII, thereby further amplifying the coagulation system.

Tertiary hemostasis is characterized by the formation of plasmin, which is the main enzyme responsible for fibrinolysis. At the same time as the coagulation cascade is activated, tissue plasminogen activator is released from endothelial cells. Tissue plasminogen activator binds to plasminogen within the clot, converting it into plasmin. Plasmin lyses both fibrinogen and fibrin in the clot, releasing fibrin and fibrinogen degradation products.

Finally, fibrin is digested by the fibrinolytic system, the major components of which are plasminogen and tissue-type plasminogen activator (tPA). Both of these proteins are incorporated into polymerizing fibrin, where they interact to generate plasmin, which, in turn, acts on fibrin to dissolve the preformed clot.

The fibrinolytic system is, in turn, regulated by three serine proteinase inhibitors, namely, antiplasmin, plasminogen activator inhibitor-1 (PAI-1), and plasminogen activator inhibitor-2 (PAI-2). Plasma D-dimers are generated when the endogenous fibrinolytic system degrades fibrin. They consist of two identical subunits derived from two fibrin molecules. Unlike fibrinogen degradation products, which are derived from fibrinogen and fibrin, D-dimers are a specific cross-linked fibrin derivative

The process of fibrin deposition is limited by mechanisms of the natural anticoagulant system. The maintenance of adequate blood flow and the regulation of cell surface activity limit the local accumulation of activated blood coagulation enzymes and complexes. Antithrombin (AT) is a plasma protein member of the serpin (serine protease inhibitor) family that inhibits the activities of all of the activated coagulation enzymes. The inhibitory effect of AT is increased several thousand-fold by binding to heparin. Protein C is a vitamin K-dependent protein that proteolyses factor Va and factor VIIIa to inactive fragments. Protein C binds to an endothelial cell protein C receptor (EPCR) and is activated by thrombin bound to thrombomodulin, another endothelial cell membrane-based protein, in a reaction that is modulated by a cofactor, protein S. Tissue factor pathway inhibitor is a lipoprotein-associated plasma protein that forms a quaternary complex with tissue factor, factor VIIa, and factor X, thereby inhibiting the extrinsic coagulation pathway.

2. Hemostatic Mechanism

The following is a description of the ways in which the hemostatic material of the invention contributes to achieving hemostasis:

a) Hemostasis Through Physical Path

When soluble hemostatic material of this invention contacts blood it stimulates a clotting cascade, which increases its concentration and viscosity, so the flow speed of blood is decreased. Meanwhile, soluble hemostatic material expands after it absorbs water and covers the wound surfaces, some part of the material is dissolved to form a viscous body and clog the end of the capillary blood vessels.

b) Hemostasis Through Chemical Path

The term “Hemostasis through chemical path” means that when soluble hemostatic material in this invention contact platelets, absorption and coagulation occur at an increased rate.

c) Hemostasis Through Physiology Path

The term “Hemostasis through physiology path” means that the hemostatic material of this invention can activate the coagulation factors in the human body and boost the formation of thrombin so as to generate hemostasis efficacy. Coagulation factor is the key factor to activate the endogenous coagulation system as well as the external coagulation system. It is already known that some coagulation factors bring positive electricity; therefore, they could be generally activated by a substance with negative electricity. Because the hemostatic material is water-soluble, it can generate large quantities of negative electricity after it is dissolved in water to activate the coagulation factors.

2. Hemostatic Material

The preferred embodiments provide compositions and materials that react with the hemostatic system to treat or prevent bleeding. In particular, the compositions and materials of preferred embodiments result in coagulation of blood. Effective delivery of hemostatic agents to wounds is desirable in the treatment of injuries characterized by bleeding, as well as in surgical procedures where the control of bleeding can become problematic, e.g., large surface areas, heavy arterial or venous bleeding, oozing wounds, and organ laceration/resectioning. The compositions and materials of preferred embodiments can possess a number of advantages in delivery of hemostatic agents to wounds, including but not limited to ease of application and removal, bioadsorption potential, antigenicity, and tissue reactivity.

Depending upon the nature of the wound and the treatment method employed, the devices of preferred embodiments can be fabricated in various forms. For example, a puff, fleece, gel, powder or sponge form can be preferable for controlling the active bleeding from artery or vein, or for controlling internal bleeding during laparoscopic procedures. In neurosurgery, where oozing brain wounds are commonly encountered, a sheet form of the hemostatic material can be preferred. Likewise, in oncological surgery, especially of the liver, it can be preferred to employ a sheet form or sponge form of the hemostatic material, which is placed in or on the tumor bed to control oozing. In dermatological applications, a sheet form can be preferred. In closing punctures in a blood vessel, a puff or fleece form is generally preferred. A suture form, especially a microsuture form, can be preferred in certain applications. Despite differences in delivery and handling characteristics of the various forms, the devices are each effective in deploying hemostatic agents to an affected site and rapidly initiating hemostatic plug formation through platelet adhesion, platelet activation, and blood coagulation.

The hemostatic material of the invention is comprised of a compound which has the structural formula shown below: 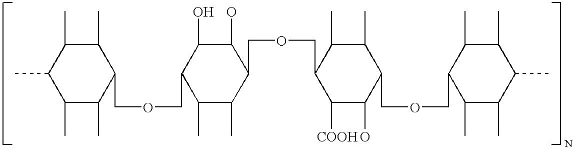
wherein n is 2-20,000, and preferably where n is between about 8,000-12,000 or preferably where n is between about 400-600.

The soluble hemostatic material is made by chemical treatment of plant fiber. The untreated plant fiber can absorb water, but is insoluble. After being treated by the process of the invention, its physical and chemical properties are changed significantly so that the resulting material is soluble in water and body fluids. It can be used both inside and outside the body to stop bleeding. When utilized in biological systems the soluble hemostatic material of this invention: absorbs water and expands, then the structure is dismantled and changes to a kind of transparent gel, and finally the material dissolves completely. The material of the invention increases hemostatic efficacy by at least three mechanisms: physical, chemical and physiological each of which are discussed below at greater length. In particular the material activates the blood-coagulation factors to boost the formation of thrombin, and the material absorbs water from the blood and expands to form colloid. Application of the material increases the viscosity of blood, blood flow speed is reduced, and the colloid clogs the opening of the blood vessel through which bleeding is taking place. Because the soluble hemostatic material activates the blood-coagulation factors and boosts the formation of thrombin, it is notably effective for patients with blood-coagulation obstructions or defects.

The hemostatic material can be provided in the form of a sheet of a pre-selected size. Alternatively, a larger sheet of hemostatic material can be cut or trimmed to provide a size and shape appropriate to the wound. Although the hemostatic material is bioabsorbable, in cutaneous or topical applications it is preferably removed from the wound after a satisfactory degree of hemostasis is achieved. When the hemostatic fabric is employed in internal applications, it is preferably left in place to be absorbed by the body over time. Such hemostatic fabrics are particularly well suited for use in the treatment of oozing wounds.

The soluble hemostatic material can be used both for a broad range of uses including clinical and for first aid. It can advantageously and easily be use in hostile environments where simple and effect means for stopping the flow of blood or body fluids is desired (e.g., battleground situations). The hemostatic material may be soluble and may be is used in the form of fabric material, such as a gauze material, and can be used on wound surfaces under pressure. The material can be provided free of any medications, if desired, or may contain desired medications for particular purposes.

The hemostatic material is suitable for use in both surgical applications as well as in field treatment of traumatic injuries. For example, the material is suitable for use in vascular surgery where bleeding is particularly problematic. The material is suitable for use in cardiac surgery where multiple vascular anastomoses and cannulation sites, complicated by coagulopathy induced by extracorporeal bypass, can result in bleeding that can only be controlled by topical hemostats. The material is suitable to produce rapid and effective hemostasis during spinal surgery, where control of osseous, epidural, and/or subdural bleeding or bleeding from the spinal cord is not amenable to sutures or cautery, can minimize the potential for injury to nerve roots and reduce the procedure time. The material is suitable for use in liver surgery, for example, in live donor liver transplant procedures or in the removal of cancerous tumors, where there is a substantial risk of massive bleeding. The material is suitable for use as an effective hemostatic material which can significantly enhance patient outcome in such procedures. Even in those situations wherein bleeding is not massive, the material is suitable for use as an effective hemostatic material, for example, in dental procedures such as tooth extractions, or for abrasions, burns, and the like. The material is suitable for use in neurosurgery, where oozing wounds are common and are difficult to treat.

The nature of soluble hemostatic material of this invention may include any combination of the following attributes:

a) Water-Solubility

The known prior art cellulose fiber materials contain hydrophilic hydroxyamino-, but there exist large quantities of hydrogen bonds among the molecules and the degree of crystallinity is high. Thus, the material can not be dissolved in water. During the processing according to the invention, the material is chemically changed so that:
i) The degree of polymerization is decreased, as is the dispersion force and inductive capacity.
ii) Hydrophilic radical groups are induced to widen the space between the molecules and destroy the hydrogen bonds inside the molecules.
iii) The degree of crystallinity is decreased, amorphism zone is enlarged, orientation force between molecules is decreased, and it is possible that water molecules form molecular compounds in tiny packs.
   From the point of view of thermodynamics, free energy of mixing between the molecules of the hemostatic material and water molecules is below zero, solubility difference is less than 1.7-2.0, so dissolution happens. The dissolution process of hemostatic material by water is: it first absorbs water and expands, unbinding of the structure then takes place and the material is transformed to transparent gel, finally it is dissolved completely.

b) Absorbability to Water and Polarizable Medium

If the speed of absorption of the hemostatic material to water and polarizable medium is high, the amount of absorption is large. This is helpful for hemostasis.

3. Method for Making Soluble Hemostatic Material

The method for making the soluble hemostatic material comprised of a compound with the following structural formula: 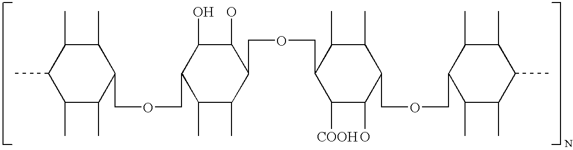
wherein n is 2-20000, comprises the steps of:
a) Placing sodium hydroxide, sodium carbonate, sodium hypochlorite in to the internal bladder of a reaction vessel, then adding in an appropriate amount of pure water and stirring until the ingredients are dissolved. Pouring ethyl alcohol (preferably about 95% ethyl alcohol) in to solution in the internal bladder and mixing. Turning on a heater and keeping the temperature of the internal bladder above 20° C., and preferably between about 25° C. and about 28° C. and holding at the desired temperature for a period of time, preferably for about 10 hours.
b) Placing the raw material to be chemically treated, preferably degreased and bleached plant fiber in the form of gauze into the mixed solution in the reaction vessel. Maintaining the temperature of the external body above 20° C., and preferably about 30° C.±3° C., and maintaining the temperature of the internal bladder between about 20 and about 30° C., and preferably about 26° C.±1° C.
c) Decreasing the temperature of the internal bladder to about 20° C.±3° C., and beginning to rotate the reaction vessel for a period of time, preferably about five hours.
d) Allowing cold water to move into the internal bladder, after a period of time the temperature will drop to below 20° C., and preferably to about 5° C.±3° C. Allow the solution to react at this decreased temperature for a period of time, preferably about one hour.
e) Adding an appropriate amount of alcohol, preferably 95% ethyl alcohol, and an appropriate amount of choloractic acid, into the reaction vessel. After 30 minutes the temperature in the internal bladder will go up to a temperature above 20° C., preferably the temperature will move from about 5° C.±3° C. to about 41° C.±3° C. Add an appropriate amount of hydrogen peroxide. Decrease the temperature below 40° C., preferably to 32° C.±3° C., allow the reaction to continue for a period of time, preferably about 1.5 hours.
f) Put the material from the reaction vessel into a container, preferably a stainless steel tub. Add an appropriate amount of alcohol, preferably 70% ethyl alcohol, stir and rise, then dry it up, preferably by centrifugal dewatering.
g) Put the material obtained as above into another container, preferably made of stainless steel, with an appropriate amount of a selected alcohol, preferably 70% ethyl alcohol, then counteract it by adding an acid, preferably Hydrochloric acid, to solution to achieve the desired pH value, preferably a pH value of about 7±0.5.
h) Take out the material and allow it to dry. Preferably one would treat the material one more time or many times as described as above in another container until the solution becomes clear. Allow the material to dry. Optionally one may iron the material to make it flat.
   4. Use of Additional Hemostatic Agents

Other suitable hemostatic agents that can be employed in preferred embodiments include, but are not limited to, clotting factor concentrates, recombinant Factor VIIa, alphanate FVIII concentrate, bioclate FVIII concentrate, monoclate-P FVIII concentrate, haemate P FVIII, von Willebrand factor concentrate, helixate FVIII concentrate, hemophil-M FVIII concentrate, humate-P FVIII concentrate, Hyate-C® Porcine FVIII concentrate, koate HP FVIII concentrate, kogenate FVIII concentrate, recombinate FVIII concentrate, mononine FIX concentrate, and fibrogammin P FXIII concentrate. Such hemostatic agents can be applied to the hemostatic material of this invention in any suitable form (powder, liquid, in pure form, in a suitable excipient, on a suitable support or carrier, or the like).

A single hemostatic agent or combination of hemostatic agents can be employed. Preferred loading levels for the hemostatic agent on the hemostatic material can vary, depending upon, for example, the nature of the selected material and hemostatic agent, the form of the material, and the nature of the wound to be treated. However, in general in the case of hemostatic gauze, a weight ratio of hemostatic agent to hemostatic gauze of from about 0.001:1 or lower to about 2:1 or higher is generally preferred. More preferably, a weight ratio of additional hemostatic agent to material of from about 0.05:1 or lower to about 2:1 or higher is generally preferred. More preferably, a weight ratio of from about 0.06:1, 0.07:1, 0.08:1, 0.09:1, 0.10:1, 0.15:1, 0.20:1, 0.25:1, 0.30:1, 0.35, 0.40:1, 0.45:1, 0.50:1, 0.55:1, 0.60:1, 0.65:1, 0.70:1, 0.75:1, 0.80:1, 0.85:1, 0.90:1, or 0.95:1 to about 1:1, 1.1:1, 1.2:1, 1.3:1, 1.4:1, or 1.5:1 is employed, although higher or lower ratios can be preferred for certain embodiments.

5. Use of Auxiliary Substances in Preparing Hemostatic Materials

In certain embodiments, it can be desirable to utilize collagen, natural cotton cellulose, pure plant fiber, silk, rayon or nylon as a hemostatic material alone or in combination with one or more hemostatic agents to accelerate clotting. Other substances that can be utilized include thrombin, fibrinogen, hydrogels, and oxidized cellulose. Other auxiliary substances can also be employed, as will be appreciated by one skilled in the art.

6. Multifunctional Hemostatic Materials

In addition to effectively delivering a hemostatic agent to a wound, the hemostatic materials of preferred embodiments can deliver other substances as well. In a particularly preferred embodiment, such substances include medicaments, pharmaceutical compositions, therapeutic agents, and/or other substances producing a physiological effect. The substances can be deposited on the hemostatic material by any other suitable method as is known in the art for depositing a material on a material or incorporating an agent into a material

Any suitable medicament, pharmaceutical composition, therapeutic agent, or other desirable substance can be incorporated into the material of preferred embodiments. Preferred medicaments include, but are not limited to, anti-inflammatory agents, anti-infective agents, anesthetics, immunosuppressive agents and chemotherapy agents.

Suitable anti-inflammatory agents include but are not limited to, nonsteroidal anti-inflammatory drugs (NSAIDs) such as aspirin, celecoxib, choline magnesium trisalicylate, diclofenac potasium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

Anti-infective agents include, but are not limited to, anthelmintics (mebendazole), antibiotics including aminoclycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim.

Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine.

Chemotherapy agents include, but are not limited to, adriamycin, alkeran, Ara-C, BiCNU, busulfan, CCNU, carboplatinum, cisplatinum, cytoxan, daunorubicin, DTIC, 5-FU, fludarabine, hydrea, idarubicin, ifosfamide, methotrexate, mithramycin, mitomycin, mitoxantrone, nitrogen mustard, taxol, velban, vincristine, VP-16, gemcitabine (gemzar), herceptin, irinotecan (camptosar, CPT-11), leustatin, navelbine, rituxan, STI-571, taxotere, topotecan (hycamtin), xeloda (capecitabine), and zevelin.

A variety of other medicaments and pharmaceutical compositions are suitable for use in preferred embodiments. These include cell proliferative agents such as tretinoin, procoagulants such as dencichine (2-amino-3-(oxalylamino)-propionic acid), and sunscreens such as oxybenzone and octocrylene.

Human epidermal growth factor (hEGF) can also be preferred for certain embodiments. This small molecular weight peptide is a mitogenic protein and is critical for skin and epidermal regeneration. It is a small 53 amino acid residue long protein with 3 disulfide bridges. The epidermal growth factor can be used as produced, or can be polymerized prior to use in preferred embodiments. Presence of hEGF can have a positive effect upon skin healing and regeneration.

Other substances which can be used in preferred embodiments can include, or be derived from, traditional medicaments, agents, and remedies which have known antiseptic, wound healing, and pain relieving properties. These agents include, but are not limited to Sanqi (Radix Notoginsent). Another such agent is Dahuang (Radix Et Rhizoma Rhei). One of its compounds has anti-inflammatory effect and can also effectively reduce soft tissue edema. The compound is Emodin. Zihuaddng (Herba Violae), which has been used as an antibiotic agent.

Baiji (Rhizoma Bletillae) has been used as a hemostatic agent and also to promote wound healing for years. It contains the following substances: (3,3′-dihydroxy-2′,6′-bis(p-hydroxybenzyl)-5-methoxybibenzy-1); 2,6-bis(p-hydroxybenzyl)-3′,5-dimethoxy-3-hydroxy-bibenzyl); (3,3′-dihydroxy-5-methoxy-2,5′,6-tris(p-hydroxy-benzyl)bibenzyl; 7-dihydroxy-1-p-hydroxybenzyl-2-methoxy-9,10-dihydro-phenanthrene); (4,7-dihydroxy-2-methoxy-9,10-dihydroxyphenanthrene); Blestriarene A (4,4′-dimethoxy-9,9′,10,10′-tetrahydro[1,1′-biphenanthrene]-2,2′,7,7′-tetrol); Blestriarene B (4,4′-dimethoxy-9,10-dihydro[1,1′-biphenanthrene]-2-,2′,7,7′-tetrol); Batatasin; 3′-O-Methyl Batatasin; Blestrin A(1); Blestrin B(2); Blestrianol A (4,4′-dimethoxy-9,9′,10,10′-tetrahydro]-1′,3-biphenanthrene]-2,2′,7,7′-tetraol); Blestranol B (4′,5-dimethoxy-8-(4-hydroxybenzyl)-9,9′,10,10′-tetrahydro-[1′,3-biphenanthrene]-2,2′,7,7′-tetraol-); Blestranol C (4′,5′-dimethoxy-8-(4-hydroxybenzyl)-9,10-dihydro-[1′,3-biphenanthrene]-2,2′,7,7′-tetraol); (1,8-bi(4-hydroxybenzyl)-4-methoxy-phenanthrene-2,7-diol); 3-(4-hydroxybenzyl)-4-methoxy-9,10-dihydro-phenanthrene-2,7-diol; (1,6-bi(4-hydroxybenzyl)-4-methoxy-9,10-dihydro-phenanthrene-2,-7-diol; (1-p-hydroxybenzyl-4-methoxyphenanthrene-2,7-diol); 2,4,7-trimethoxyphenanthrene; 2,4,7-trimethoxy-9,10-dihydrophenanthrene; 2,3,4,7-tetramethoxyphenanthrene; 3,3′,5-trimethoxy-bibenzyl; 3,5-dimethoxybibenzyl; and Physcion.

Rougui (Cortex Cinnamoni) has pain relief effects. It contains the following substances: anhydrocinnzeylanine; anhydrocinnzeylanol; cinncassiol A; cinnacassiol A monoacetate; cinncassiol A glucoside; cinnzeylanine; cinnzeylanol; cinncassiol B glucoside; cinncassiol C.sub.1; cinncassiol C.sub.1 glucoside; cinncassiol C.sub.2; cinncassiol C.sub.2; cinneassiol D.sub.1; cinncassiol D.sub.1 glucoside; cinncassiol D.sub.2; cinncassiol D.sub.2 glucoside; cinncassiol D.sub.3; cinncassiol D.sub.4; cinncassiol D.sub.4 glucoside; cinncassiol E; lyoniresinol; 3.alpha.-O-B-D-glucopyranoside; 3,4,5-trimethoxyphenyl 1-O-.beta.-D-apiofuranosyl-(1.fwdarw.6)-.beta.-D-glucopyranoside; (.+−.)-syringaresinol; cinnamic aldehyde cyclic glycerol 1,3 acetals; epicatechin; 3′-O-methyl-(−)-epicatechin; 5,3′-di-O-methyl-(−)-epicatechin-; 5,7,3′-tri-O-methyl-(−)-epicatechin, 5′-O-methyl-(+)-catechin; 7,4′-di-O-methyl-(+)-catechin; 5,7,4′-tri-O-methyl-(+)-catechin; (−)-epicatechin-3-O-.beta.-D-glucopyranoside; (−)-epicatechin-8-C-.beta.-D-glucopyranoside; (−)-epicatechin-6-C-.beta.-D-glucopyranoside; procyanidin; cinnamtannin A.sub.2, A.sub.3, A.sub.4; (−)-epicatechin; procyanidins B-1, B-2, B-5, B-7, C-1; proanthocyanidin; proanthocyanidin A-2; 8-C-.beta.-D-glucopyranoside; procyanidin B-2 8-C-.beta.-D-glycopyranoside; cassioside[(4s)-2,4-dimethyl-3-(4-hydroxy-3-hydroxymethyl-1-butenyl)-4-(.beta.-D-glucopyranosyl)methyl-2-cyclohexen-1-one]; 3,4,5-trimethoxyphenyl-.beta.-D-apiofuranosyl-1(1.fwdarw.6) -.beta.-D-glucopyranoside; coumarin; cinnamic acid; procyanidin; procyanidin B.sub.2; cinnamoside[(3R)-4-{(2′R,4′S)-2′-hydroxy-4′-(.beta.-1-D-apiofuranoxy-(1.fwdarw.6)-.beta.-D-glucopyranosyl)-2′,6′,6′-trimethyl-cyclohexylidene}-3-buten-2-one]; cinnamaldehyde; 3-2(hydroxyphenyl)-propano-ic acid; O-glucoside; cinnaman A.sub.2; P, S, Cl, K, Ca, Ti, Mn, Fe, Cu, Zn, Br, Rb, Sr, and Ba.

Other substances that can be incorporated into the hemostatic agents of preferred embodiments include various pharmacological agents, excipients, and other substances well known in the art of pharmaceutical formulations. Other pharmacological agents include, but are not limited to, antiplatelet agents, anticoagulants, ACE inhibitors, and cytotoxic agents. These other substances can include ionic and nonionic surfactants (e.g., Pluronic™, Triton™), detergents (e.g., polyoxyl stearate, sodium lauryl sulfate), emulsifiers, demulsifiers, stabilizers, aqueous and oleaginous carriers (e.g., white petrolatum, isopropyl myristate, lanolin, lanolin alcohols, mineral oil, sorbitan monooleate, propylene glycol, cetylstearyl alcohol), emollients, solvents, preservatives (e.g., methylparaben, propylparaben, benzyl alcohol, ethylene diamine tetraacetate salts), thickeners (e.g., pullulin, xanthan, polyvinylpyrrolidone, carboxymethylcellulose), plasticizers (e.g., glycerol, polyethylene glycol), antioxidants (e.g., vitamin E, vitamin K, vitamin C etc.), buffering agents, flexible agents (e.g., silicon), antibiotics, low-grade antibiotics (e.g., tetracycline, silver, etc.) and the like.

7. Alternative Forms of Hemostatic Materials

While it is generally preferred to apply the hemostatic material (for example, a hemostatic fabric, sponge, puff, matrix, or powder prepared as described above, or another form) directly to the wound, in certain embodiments it can be preferred to incorporate the hemostatic material into a wound dressing including other components.

To ensure that the hemostatic material remains affixed to the wound, a suitable adhesive can be employed, for example, along the edges or a side of the hemostatic fabric, sponge or puff. Although any adhesive suitable for forming a bond with skin or other tissue can be used, it is generally preferred to use a pressure sensitive adhesive. Pressure sensitive adhesives are generally defined as adhesives that adhere to a substrate when a light pressure is applied but leave no residue when removed. Pressure sensitive adhesives include, but are not limited to, solvent in solution adhesives, hot melt adhesives, aqueous emulsion adhesives, calenderable adhesive, and radiation curable adhesives. Solution adhesives are preferred for most uses because of their ease of application and versatility. Hot melt adhesives are typically based on resin-tackified block copolymers. Aqueous emulsion adhesives include those prepared using acrylic copolymers, butadiene styrene copolymers, and natural rubber latex. Radiation curable adhesives typically consist of acrylic oligomers and monomers, which cure to form a pressure sensitive adhesive upon exposure to ultraviolet lights.

The most commonly used elastomers in pressure sensitive adhesives include natural rubbers, styrene-butadiene latexes, polyisobutylene, butyl rubbers, acrylics, and silicones. In preferred embodiments, acrylic polymer or silicone based pressure sensitive adhesives are used. Acrylic polymers generally have a low level of allergenicity, are cleanly removable from skin, possess a low odor, and exhibit low rates of mechanical and chemical irritation. Medical grade silicone pressure sensitive adhesives are preferred for their biocompatibility.

Amongst the factors that influence the suitability for a pressure sensitive adhesive for use in wound dressings of preferred embodiments are the absence of skin irritating components, sufficient cohesive strength such that the adhesive can be cleanly removed from the skin, ability to accommodate skin movement without excessive mechanical skin irritation, and good resistance to body fluids.

In preferred embodiments, the pressure sensitive adhesive comprises a butyl acrylate. While butyl acrylate pressure sensitive adhesives are generally preferred for many applications, any pressure sensitive adhesive suitable for bonding skin can be used. Such pressure sensitive adhesives are well known in the art.

As discussed above, the hemostatic materials of preferred embodiments generally exhibit good adherence to wounds such that an adhesive, for example, a pressure sensitive adhesive, is generally not necessary. However, for ease of use and to ensure that the hemostatic material remains in a fixed position after application to the wound, it can be preferable to employ a pressure sensitive adhesive.

While the hemostatic puffs, powder, gel, fabrics and other hemostatic materials of preferred embodiments generally exhibit good mechanical strength and wound protection, in certain embodiments it can be preferred to employ a backing or other material on one side of the hemostatic material. For example, a composite including two or more layers can be prepared, wherein one of the layers is the hemostatic material and another layer is, e.g., an elastomeric layer, gauze, vapor-permeable film, waterproof film, a woven or nonwoven fabric, a mesh, or the like. The layers can then be bonded using any suitable method, e.g., adhesives such as pressure sensitive adhesives, hot melt adhesives, curable adhesives, application of heat or pressure such as in lamination, physical attachment through the use of stitching, studs, other fasteners, or the like.

Other components can be combined with the hemostatic materials for use in wound dressings as are known in the art, such as preservatives, stabilizers, dyes, buffers, alginate pastes or beads, hydrocolloid pastes or beads, hydrogel pastes or beads, as well as medicaments and other therapeutic agents as described above.

In another preferred embodiment the soluble hemostatic material can be combined with water in a mixture to produce a hemostatic gel. Bleeding can be treated with direct application of the gel. The gel can be used inside and outside the body. As described above, when combined with a liquid, the hemostatic material of this invention absorbs the liquid and expands, then the structure is dismantled and changes to a kind of transparent gel. Later, the material can dissolve completely after being applied to bleeding.

A number of liquids can be combined with the soluble hemostatic material. In one of the preferred embodiments the liquid can be distilled water. In yet another preferred embodiment, the liquid can contain the following elements in the approximate amounts listed below.

| | | |
|---|---|---|
| Carbon | 600-1000 | ppm |
| Sulfur | 500-900 | ppm |
| Oxygen | 200-350 | ppm |
| Calcium | 150-200 | ppm |
| Iron | 100-200 | ppm |
| Sodium | 100-150 | ppm |
| Aluminum | 80-150 | ppm |
| Potassium | 20-70 | ppm |
| Magnesium | 20-70 | ppm |
| Hydrogen | 20-70 | ppm |
| Nitrogen | 10-50 | ppm |
| Silica | 5-40 | ppm |
| Phosphorus | 1-5 | ppm |
| Titanium | 0.5-2 | ppm |
| Boron | 0.5-2 | ppm |
| Manganese | 0.5-2 | ppm |
| Lithium | 0.5-2 | ppm |
| Silver | 0.5-2 | ppm |
| Zinc | 0.5-2 | ppm |
| Copper | 0.5-2 | ppm |

In still another preferred embodiment, the liquid can contain the following elements in the approximate amounts listed below.

| | | |
|---|---|---|
| Carbon | 873 | ppm |
| Sulfur | 687 | ppm |
| Oxygen | 286 | ppm |
| Calcium | 183 | ppm |
| Iron | 145 | ppm |
| Sodium | 120 | ppm |
| Aluminum | 116 | ppm |
| Potassium | 44 | ppm |
| Magnesium | 39 | ppm |
| Hydrogen | 38 | ppm |
| Nitrogen | 25 | ppm |
| Silica | 14 | ppm |
| Phosphorus | 2.1 | ppm |
| Titanium | 1 | ppm |
| Boron | 1 | ppm |
| Manganese | 1 | ppm |
| Lithium | 1 | ppm |
| Silver | 1 | ppm |
| Zinc | 1 | ppm |
| Copper | 1 | ppm |

In addition to the elements listed above, the liquid can contain natural combinations of the following trace elements in amounts less than 1 part per million: Antimony, Arsenic, Barium, Beryllium, Bismuth, Bromide, Cadmium, Cesium, Chlorine, Chromium, Cobalt, Dysprosium, Erbium, Europium, Gadolinium, Gallium, Germanium, Gold, Hafnium, Holmium, Indium, Iridium, Lanthanum, Lead, Lutetium, Molybdenum, Mercury, Neodymium, Nickel, Niobium, Osmium, Palladium, Platinum, Praseodymium, Rhenium, Rhodium, Rubidium, Ruthenium, Samarium, Scandium, Selenium, Stontium, Tantalim, Tellurium, Terbium, Thallium, Thulium, Tin, Tungsten, Uranium, Vanadium, Ytterbium, Yttrium, and Zirconium. This solution produces a supplement that can assist immune system function.

In yet another preferred embodiment, a liquid can further be combined with B1, B2, B6, B12, Folic Acid, D3, Boron, Manganese, Potassium, Calcium, Magnesium, and Zinc. This addition can provide a benefits not only to the wound but to the body in general, including, but not limited to providing nutritional controls toward the proper maintenance of levels of homocysteine (dangerous cholesterol) in the blood; enhancing digestive functions; reducing bone fractures; helping Alzheimer's disease; sustaining brain function and supporting cognitive activity and healthy nerve function; enhancing circulation and flood formation; helping to sustain all growth functions and processes; contributing to healthy skin; assisting cellular rejuvenation; sustaining cellular replication and the synthesis of RNA and DNA; strengthening and supporting several levels of the immune system; supporting the maintenance of bones, teeth and skin; supporting cardiovascular health; and reducing nervousness and supports positive mental function.

Proportions of liquid to water may vary. For example, in one of the preferred embodiments of the invention four—2 cm² pieces of gauze hemostatic material can be combined with one ounce of liquid. Explanation of the production of a 2 cm² piece of gauze hemostatic material is described below in Example 1. In other preferred embodiments, the number of 2 cm² pieces of gauze hemostatic material combined with one ounce of liquid can be greater of smaller than four, such as three, six, or eight.

Depending on the nature of the wound, the means for delivery of the hemostatic gel may vary. In one of the preferred embodiments the gel may be loaded into a syringe or other delivery apparatus (e.g., squeezable tube) and delivered to a wound. The syringe could be inserted into the entrance of wounds otherwise difficult to access, such as the entrance of a narrow bodily cavity. The syringe could have a memory tube. Alternatively and additionally, disposable syringes could be used as well as syringes of a variety of sizes depending on the nature of the wound. In addition, the syringe could be a preloaded, pre-sterilize syringe.

In yet other preferred embodiments the means for delivery of the hemostatic gel can be pre-applied to a bandage for application to exterior wounds or included in a squeezable tube for multi-purpose application and ease of storage and sale. Alternatively and additionally, the gel could be included in a variety of kits designed for first-aid and emergency situations. In each of these preferred embodiments the gel can include a therapeutic agent, such as an anti-inflammatory agent or disinfectant, as described above. Other embodiments of the present invention embrace the use of a variety of means for delivery of the hemostatic gel not herein listed but which would be appreciated by one of ordinary skill in the art.

The following examples will describe this invention in detail, but these examples shall not be construed as limiting the scope of this invention.

### EXAMPLE 1

The preferred hemostatic material of the invention comprise a soluble hemostatic material with the following structural formula: 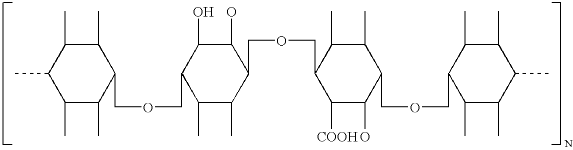
wherein n is 2-20000. The preferred method for making the preferred material of the invention comprises the steps of:

1) Activating Treatment:
a) Placing two liters of sodium hydroxide, two liters of sodium carbonate, and one liter sodium hypochlorite in to the internal bladder of a reaction vessel, then adding in an appropriate amount of pure water and stirring until the ingredients are totally dissolved and a pH value of 8 to 9.5 is achieved. Then, pour 60 liters of 95% ethyl alcohol in to the internal bladder and mix. Then turn on the stainless steel heater and keep the temperature of the internal bladder at 25 to 28° C. and hold for 10 hours.
b) Put 80 meters clinical use gauze into the mixed solution in the reaction vessel. At this point, the temperature of the external body should be 30° C.±3° C., the temperature of the internal bladder should be 26° C.±1° C.
c) Decrease the temperature of the internal bladder to 20° C.±3° C., and begin to rotate the reaction vessel for three to five hours.
d) Allow cold water from a refrigerator to move into the internal bladder with a temperature of 20° C.±3 C, after 30 minutes the temperature will drop to 5° C.±3° C. Allow this reaction to occur for one hour.

2) Oxidizing Treatment
a) Add 60 liters of 95% ethyl alcohol and 12 bottles of choloractic acid into the reaction vessel, then let in water with the temperature at 45° C. After 30 minutes the temperature in the internal bladder will go up from 5° C.±3° C. to 41° C.±3° C. Add one bottle of hydrogen peroxide, decrease the temperature to 32° C.±3° C., allow the reaction to continue for 1.5 hours

3) Rinsing and Drying Up
a) Put the gauze form the reaction vessel into a stainless tell tub, add in 60 kg 70% ethyl alcohol, stir and rise, then dry it up by centrifugal dewatering.
b) Put the gauze obtained as above into another stainless steel tub with 60 kg 70% ethyl alcohol; counteract it by adding into Hydrochloric acid to achieve the pH value of 7±0.5.
c) Take out the gauze and dry it up and treat the gauze one more time or many times as described as above in another stainless steel tub until the solution becomes clear, then take out the gauze dry it up and make it flat by ironing.
d) Dry the rinsed gauze up in a dryer. Turn on the power switch, press on the drying button, the dryer begins to run and removes the unwanted ethyl alcohol form the gauze.

4) Sterilizing and Ironing Out
a) Take out the gauze and insert one end thereof into the rollers for drying and ironing, the rolling of the rollers makes the gauze go through and at the meantime dry up the gauze and iron out the gauze and so the gauze comes out flat and then is scrolled up.
b) 2 cm² gauze is cut and dipped into a cup with water, after 2-3 minutes, it appears in a viscous manner, within 2 hours, it dissolves in to a mixture with the water. Having described these aspects of the invention, it is understood that the invention provides a new kinds of soluble hemostatic fabric material and gel and it can be made in the industry simply and economically. It is also understood that the invention defined by the appended claims is not to be limited by particular details set forth in the above description, as many apparent variations thereof are possible without departing from the spirit or scope thereof.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A hemostatic gel comprising:
(a) a liquid; and
(b) a compound with the structure formula: 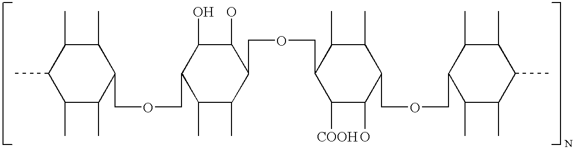
wherein N is in a range between about 2 and about 20,000;

2. The gel of claim 1, wherein the liquid is distilled water.

3. The gel of claim 1, wherein the liquid further contains of at least one element selected from the group consisting of: Carbon, present in amounts between 600 and 1000 ppm; Sulfur, present in amounts between 500 and 900 ppm, Oxygen present in amounts between 600 and 1000 ppm; Calcium present in amounts between 200 and 350 ppm; Iron present in amounts between 100 and 200 ppm; Sodium present in amounts between 100 and 150 ppm; Aluminum present in amounts between 80 and 150 ppm; Potassium present in amounts between 20 and 70 ppm; Magnesium present in amounts between 20 and 70 ppm; Hydrogen present in amounts between 20 and 70 ppm; Nitrogen present in amounts between 10 and 50 ppm; Silica present in amounts between 5 and 40 ppm; Phosphorus present in amounts between 1 and 5 ppm; Titanium present in amounts between 0.5 and 2 ppm; Boron present in amounts between 0.5 and 2 ppm; Manganese present in amounts between 0.5 and 2 ppm; Lithium present in amounts between 0.5 and 2 ppm; Silver present in amounts between 0.5 and 2 ppm; Zinc present in amounts between 0.5 and 2 ppm; Copper present in amounts between 0.5 and 2 ppm, and combinations thereof.

4. The gel of claim 1, wherein the liquid further includes at least one trace element selected from the group consisting of: Antimony, Arsenic, Barium, Beryllium, Bismuth, Bromide, Cadmium, Cesium, Chlorine, Chromium, Cobalt, Dysprosium, Erbium, Europium, Gadolinium, Gallium, Germanium, Gold, Hafnium, Holmium, Indium, Iridium, Lanthanum, Lead, Lutetium, Molybdenum, Mercury, Neodymium, Nickel, Niobium, Osmium, Palladium, Platinum, Praseodymium, Rhenium, Rhodium, Rubidium, Ruthenium, Samarium, Scandium, Selenium, Stontium, Tantalim, Tellurium, Terbium, Thallium, Thulium, Tin, Tungsten, Uranium, Vanadium, Ytterbium, Yttrium, Zirconium, and combinations thereof.

5. The gel of claim 1, wherein the liquid further includes at least one additive element selected from the group consisting of: vitamin B1, vitamin B2, vitamin B6, vitamin B12, Folic Acid, vitamin K, silver, natural silver, vitamin D3, Boron, Manganese, Potassium, Calcium, Magnesium, Zinc, and combinations thereof.

6. The hemostatic gel of claim 1, further comprising a therapeutic agent.

7. The hemostatic gel of claim 6, wherein the therapeutic agent comprises an agent selected from a list comprising: an anti-inflammatory agent, an anti-infective agent, an anesthetic, a chemotherapy agent.

8. The hemostatic gel of claim 1, further comprising a means for delivery of the hemostatic gel selected from a group consisting of: a syringe, a disposable syringe, a syringe and memory tube, a container with cotton tip applicator, a container with associated applicator, a cotton tip pipette, a squeezable tube, and a bandage having hemostatic gel pre-applied.

9. A process for preparing a hemostatic gel, the process comprising:
a) placing sodium hydroxide, sodium carbonate, sodium hypochlorite and water in to a reaction vessel;
b) placing an amount of alcohol into the reaction vessel;
c) placing the raw material to be chemically treated into the reaction vessel;
d) adding an appropriate amount of alcohol, and an appropriate amount of choloractic acid, into the reaction vessel;
e) adding an appropriate amount of hydrogen peroxide, whereby a hemostatic material is obtained; and
f) placing the hemostatic material in a liquid solution, whereby the hemostatic material is dissolved and a gel is obtained.

10. The process of claim 9, wherein the alcohol added is 95% ethyl alcohol. pure water and stirring until the ingredients are dissolved.

11. The process of claim 9, wherein step (c) further comprising the steps of:
decreasing the temperature of the solution to about 20° C.;
mixing the solution for a period of about five hours;
cooling the solution to about 5° C.; and
allowing the solution to react at about 5° C. for about one hour.

12. The process of claim 7, wherein the alcohol utilized in step (d) is 95% ethyl alcohol.

13. The process of claim 7, wherein during step (d) the solution is heated to about 41° C.

14. The process of claim 9, wherein step (e) further comprises the steps of:
decreasing the temperature to about 32° C.; and allowing the reaction to continue for a period of about 1.5 hours.

15. The process of claim 9, further comprising the steps of:
f) placing the material from the reaction vessel into a separate container;
g) adding an appropriate amount of alcohol;
h) removing excess fluid from the material;
i) adding an appropriate amount of an acid to solution to achieve the desired pH value; and
j) allowing the material to dry.

16. The process of claim 9, wherein the hemostatic material of stem (f) hemostatic gauze.

17. The process of claim 14, wherein the ratio of hemostatic gauze to liquid is selected for a group consisting of: four 2 cm² pieces of hemostatic gauze: 1 ounce of liquid, six 2 cm² pieces of hemostatic gauze: 1 ounce of liquid, and eight 2 cm² pieces of hemostatic gauze: 1 ounce of liquid.

18. The process of claim 9, further comprising: compressing the hemostatic material between a first surface and a second surface; and heating the compressed hemostatic material, whereby a dry hemostatic material is obtained.

19. A method of controlling bleeding the method comprising:
(a) adding a compound to a mixture, wherein the compound has the structural formula: 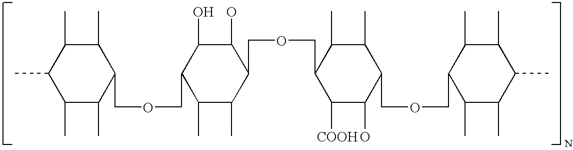
wherein N is in a range between about 2 and about 20,000.
(b) adding water to the mixture, whereby a gel is obtained.

20. The method of claim 18, wherein the hemostatic material further comprises an agent selected from the group consisting of an anti-inflammatory agent, an anti-infective agent, and an anesthetic.
